(19) United States
(12) Patent Application Publication
(10) Pub. No.: US 2005/0070856 A1
(43) Pub. Date: Mar. 31, 2005

(54) Adjustable angle eye dropper

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/884,608

(22) Filed: Jul. 1, 2004

Related U.S. Application Data
(63) Continuation-in-part of application No. 10/670,961, filed on Sep. 25, 2003.
(63) Continuation-in-part of application No. 10/728,701, filed on Dec. 5, 2003.

(51) Int. Cl. A61M 35/00; A61M 5/00

(52) U.S. Cl. 604/295

(57) ABSTRACT

A flexible elongated sealed container with a self-contained opening means fully enclosed within the container to release the substance sealed within the container is disclosed. The enclosed opening means is operated by bending the flexible elongated sealed container at or near the enclosed opening means to release the substance sealed within the flexible elongated sealed container for application. There are no loose parts that may be lost and all components are completely sealed within the container.

This application is a continuation-in-part of the prior nonprovisional U.S. patent application Ser. No. 10/670,961, filed on Sep. 25, 2003, entitled Sealed Container with Enclosed Opening Means. This application is also a continuation-in-part of the prior nonprovisional U.S. patent application Ser. No. 10/728,701, filed on Dec. 5, 2003, entitled Enclosed Opening Means.

### BACKGROUND

1. Field of the Invention

The present invention relates generally to eye droppers. More specifically, the present invention is an eye dropper with an adjustable angle applicator tip to allow convenient application of its content to the eye.

2. Description of Related Art

Conventional eye droppers are generally in the form of a small squeezable plastic container with a restricted opening formed as an applicator tip covered with a protective cover such as a screw-on cap. The medication is contained in the small squeezable plastic container. After the protective cover is removed, the eye dropper is maneuvered to position the applicator tip immediately above the eye to be applied. The plastic container is then squeezed to force a few drops of the medication out of the small squeezable plastic container and applied to the eye through the applicator tip.

An eye is a relatively small area to apply medication to. This difficulty is compounded when one is applying the medication to one's own eyes. The conventional eye dropper is difficult to aim at the eyes and requires the hand and arm to be extended and bent at an awkward position to accurately apply the medication to an eye. For a user with joint or muscular disabilities, this can be virtually impossible to perform. In addition, an elderly user who may not be as steady with his or her hands and coordination may find the application of medication to the eye with conventional eye dropper a difficult and frustrating process.

### BRIEF SUMMARY OF THE INVENTION

The present invention is an adjustable angle eye dropper with a self-contained opening means fully enclosed within the container to release the substance sealed within the container. The enclosed opening means is operated by bending the flexible elongated sealed container at or near the enclosed opening means. Once the enclosed opening means is opened, the substance sealed within the flexible elongated sealed container may be released for application. When the flexible elongated sealed container has a small cross-section such that the substance within it cannot be released due to its surface tension, a guiding member may be utilized to increase the capillary action and to overcome the surface tension of the substance to release the substance from the flexible elongated sealed container. There are no loose parts that may be lost and all components are completely sealed within the container.

### BRIEF DESCRIPTION OF THE DRAWINGS

FIG. 1 shows the preferred embodiment of the adjustable angle eye dropper.

FIG. 2 shows another embodiment of the adjustable angle eye dropper.

FIG. 3 shows another embodiment of the adjustable angle eye dropper.

FIG. 4 shows another embodiment of the adjustable angle eye dropper.

FIG. 5 shows another embodiment of the adjustable angle eye dropper.

FIG. 6 shows one embodiment of the opening means in the closed condition.

FIG. 7 shows the opening means of FIG. 6 in the open position to release the liquids in the sealed container to be released.

FIG. 8 shows one embodiment of the opening means in the closed condition.

FIG. 9 shows the opening means of FIG. 8 in the open position to release the liquids in the sealed container to be released.

FIG. 10 shows one embodiment of the opening means in the closed condition.

FIG. 11 shows the opening means of FIG. 10 in the open position to release the liquids in the sealed container to be released.

FIG. 12 shows a cross-sectional view of another embodiment of the adjustable angle eye dropper.

FIG. 13 shows a cross-sectional view of another embodiment of the adjustable angle eye dropper.

FIG. 14 shows a cross-sectional view of another embodiment of the adjustable angle eye dropper.

FIG. 15 shows a cross-sectional view of another embodiment of the adjustable angle eye dropper.

FIG. 16 shows a cross-sectional view of another embodiment of the adjustable angle eye dropper.

FIG. 17 shows a cross-sectional view of another embodiment of the adjustable angle eye dropper.

### DETAILED DESCRIPTION OF THE PREFERRED EMBODIMENT

The following description and figures are meant to be illustrative only and not limiting. Other embodiments of this invention will be apparent to those of ordinary skill in the art in view of this description.

FIG. 1 shows the preferred embodiment of the adjustable angle eye dropper. In the preferred embodiment, the adjustable angle eye dropper comprises of a flexible elongated tubular housing 1 with a sealed end 2 and an open end 3. A substance 4 is enclosed within the flexible elongated tubular housing 1 near the sealed end 2. An enclosed opening means 5 is disposed inside the flexible elongated tubular housing 1 sealing the substance 4 within the flexible elongated tubular housing 1. The enclosed opening means 5 is operated by bending the flexible elongated tubular housing 1 at or near the enclosed opening means 5. The adjustable angle eye dropper will simultaneously bent and open at the position of the enclosed opening means 5 means 5 without fracturing the flexible elongated tubular housing 1. With the flexible elongated tubular housing 1 made of an elastic plastic material such as polypropylene, after it is bent at the enclosed opening means 5, the flexible elongated housing 1 will retain its bent shape due to the stretching and deformation of the flexible elongated housing 1 during the bending and opening of the enclosed opening means 5. This will allow the applicator end 3 to be positioned at virtually any desired angle for application of the substance 4. The applicator end 3 may also be provided with an applicator tip such as a smooth soft rounded tip without any jagged edge. The applicator tip may be made of silicone rubber or plastic.

When the flexible elongated tubular housing 1 has a small cross-section such that the substance 4 within it cannot be released simply by opening the enclosed opening means 5 due to its surface tension, a guiding member 6, 7 may be utilized to increase the capillary action and to overcome the surface tension of the substance 4 to release the substance 4 from the flexible elongated tubular housing 1 as shown in FIGS. 2, 4, and 5.

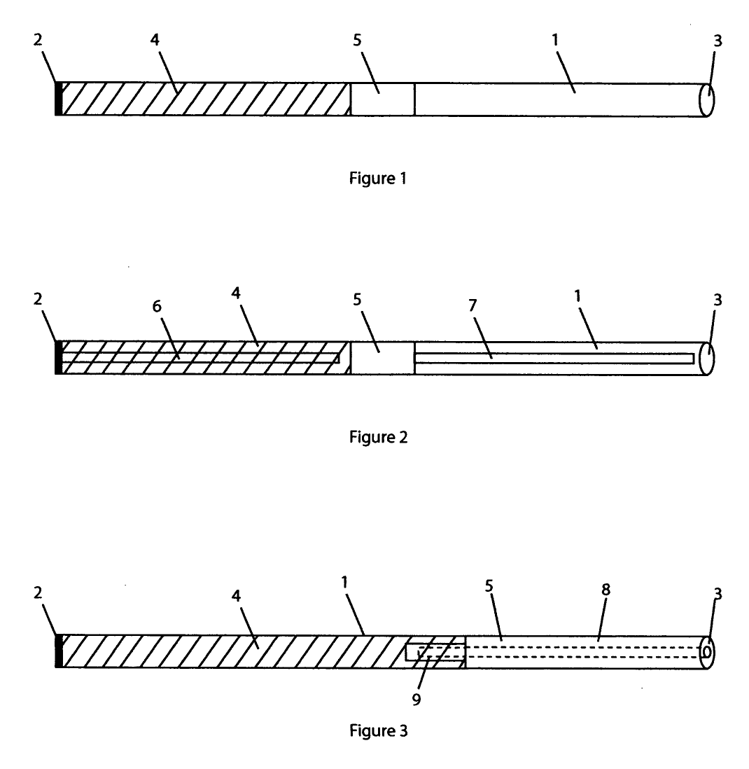

One embodiment of the enclosed opening means 5 is shown in FIG. 3 wherein the enclosed opening means 5 comprises of a cylinder 8 with an outside diameter approximately that of the inside diameter of the flexible elongated tubular housing 1 defining a small fluid path from the open end 3 of the flexible elongated tubular housing 1 to the substance 4. The end near the substance 4 has an elongated protrusion 9 that is smaller in diameter than the cylindrical body 8 of the enclosed opening means 5 and is separable from the cylindrical body 8 of the enclosed opening means 5. The elongated protrusion 9 seals the small fluid path in the cylinder 8 and prevents the substance 4 in the flexible elongated tubular housing 1 from being released through the enclosed opening means 5. When the flexible elongated tubular housing 1 is bent near the junction between the elongated protrusion 9 and the cylindrical body 8 of the enclosed opening means 5, the elongated protrusion 9 will be separated from the cylindrical body 8 and the small fluid path is exposed for the substance 4 to be released from the flexible elongated tubular housing 1 through the opening means 5.

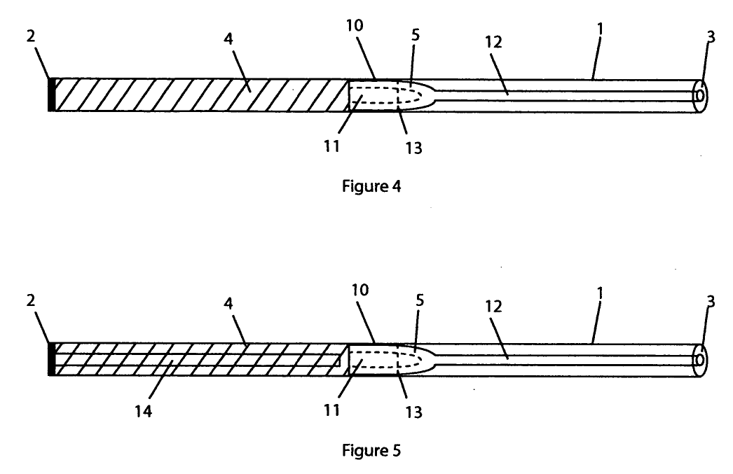

FIG. 4 shows another embodiment of the adjustable angle eye dropper. In this embodiment, the adjustable angle eye dropper comprises of a flexible elongated tubular housing 1 with a small cross-section and with a sealed end 2 and an open end 3. A substance 4 is enclosed within the flexible elongated tubular housing 1 near the sealed end 2. An enclosed opening means 5 is disposed inside the flexible elongated tubular housing 1 sealing the substance 4 within the flexible elongated tubular housing 1.

The enclosed opening means 5 comprises of a hollow cylindrical body 10 with an open end 11 toward the substance 4 and a sealed end with an elongated member 12 extending away from the hollow cylindrical body 10 towards the open end 3 of the flexible elongated tubular housing 1 and terminating near the open end 3 of the flexible elongated tubular housing 1. A frangible section 13, which may be formed by a score line on the outer surface of the hollow cylindrical body 10, is formed on the hollow cylindrical body 10 near the sealed end of the hollow cylindrical body 10.

When the flexible elongated tubular housing 1 is bent at or near the frangible section 13 of the hollow cylindrical body 10 of the opening means 5, the sealed end of the hollow cylindrical body 10 will separate from the open end 11 of the hollow cylindrical body 10 which results in a through fluid path from the substance 4 to the open end 3 of the flexible elongated tubular housing 1. Due to the small cross-section of the flexible elongated tubular housing 1, the substance 4 will not flow freely out of the flexible elongated tubular housing 1 due to surface tension of the substance 4. The substance 4 may be selectively released from the flexible elongated tubular housing 1 by squeezing the portion of the flexible elongated tubular housing 1 containing the substance 4 to force the substance 4 toward the elongated member 12. Once the substance 4 reaches the elongated member 12, the elongated member 12 will increase the capillary action of the substance 4 to overcome the surface tension of the substance 4 within the flexible elongated tubular housing 1 to allow it to freely flow out of the flexible elongated tubular housing 1.

If immediate release of the substance 4 upon opening of the opening means 5 is desired, a second elongated member 14 may be affixed to the sealed end 2 of the flexible elongated tubular housing 1 and extending to near the open end 11 of the cylindrical body 10 as shown in FIG. 5 to increase the capillary action of the substance 4 to overcome the surface tension of the substance 4 to allow the substance 4 to be immediately released from the flexible elongated tubular housing 1 upon opening of the opening means 5 without being squeezed.

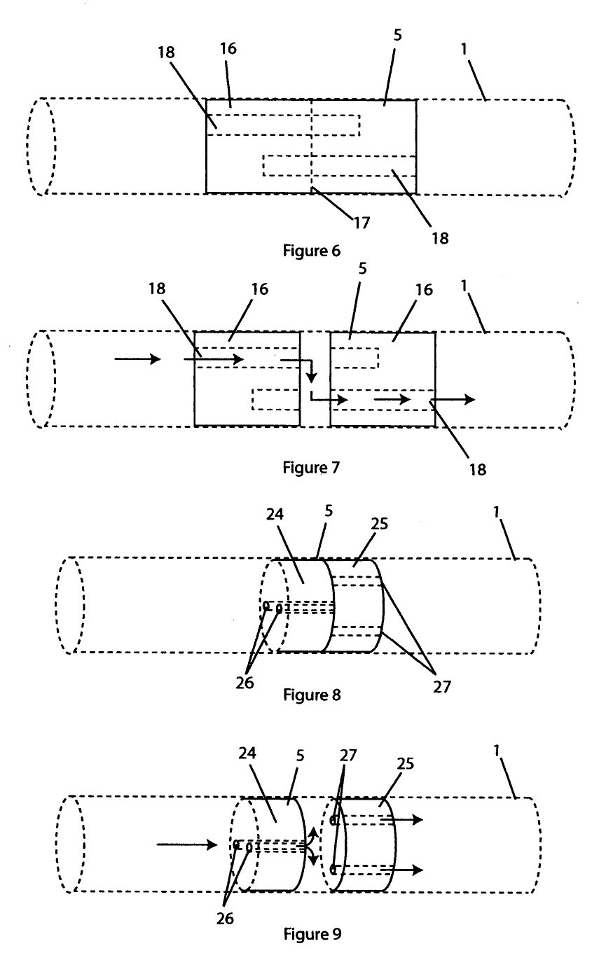

FIG. 6 shows another embodiment of the enclosed opening means 5. In this embodiment, the enclosed opening means 5 comprises of a plug 16 that is fracturable into two parts at a fracture surface 17 when it is bent. The plug 16 has one or more holes 18 extending from each end of the plug 16 pass the fracture surface 17 without passing through the other end of the plug 16. As shown in FIG. 7, upon bending of the flexible elongated tubular housing 1 at or near the plug 16, the plug 16 will separate into two sections at the fracture surface 17 and one or more fluid flow paths are formed leading from the substance 4 to the open end 3 of the flexible elongated tubular housing 1.

FIG. 8 shows another embodiment of the enclosed opening means 5 wherein the enclosed opening means 5 comprises of two circular discs 24, 25 with approximately the same cross-section as the inside diameter of the flexible elongated tubular housing 1 and with sufficient thickness to prevent them from flipping inside the flexible elongated tubular housing 1. Each circular disc 24, 25 has one or more holes 26, 27 through it positioned such that when the two circular discs 24, 25 are placed next to each other in the flexible elongated tubular housing 1, the holes 26, 27 in each circular disc will be covered by the other disc therefore all the holes 26, 27 in both circular discs 24, 25 are sealed by the other circular disc. When the flexible elongated tubular housing 1 is bent at or near the two circular discs 24, 25, the deformation of the flexible elongated tubular housing 1 will cause the two circular discs 24, 25 to separate from each other and a fluid flow path will result from the substance 4 to the open end 3 of the flexible elongated tubular housing 1 as shown in FIG. 9.

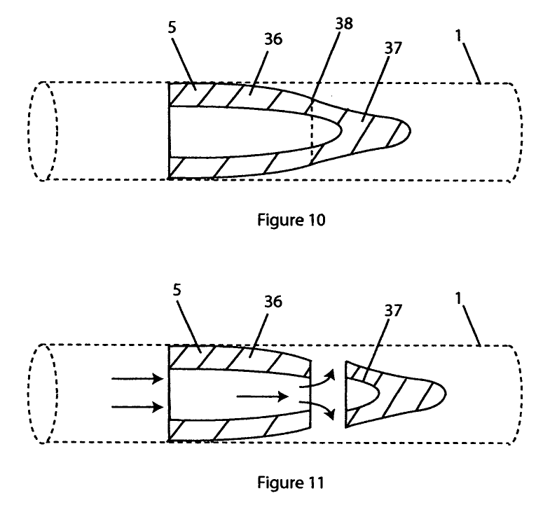

FIG. 10 shows another embodiment of the enclosed opening means 5 wherein the enclosed opening means 5 comprises of an elongated cup 36 with a diameter approximately that of the inside diameter of the flexible elongated tubular housing 1 and a length that is longer than the diameter. The closed end 37 of the elongated cup 36 is formed in an elongated protrusion with a fracture line 38 formed around the elongated protrusion positioned along the elongated cup 36 at a point where when the elongated protrusion is fractured, an opening will be formed at the closed end 37 of the elongated cup 36. The elongated cup 36 is inserted into the flexible elongated tubular housing 1 to seal the substance 4 within the flexible elongated tubular housing 1. As shown in FIG. 11, upon bending the flexible elongated tubular housing 1 near the closed end 37 of the elongated cup 36, the elongated protrusion will fracture from the elongated cup 36 at the fracture line 38 and a fluid flow path is exposed to allow the substance 4 to be released from the flexible elongated tubular housing 1.

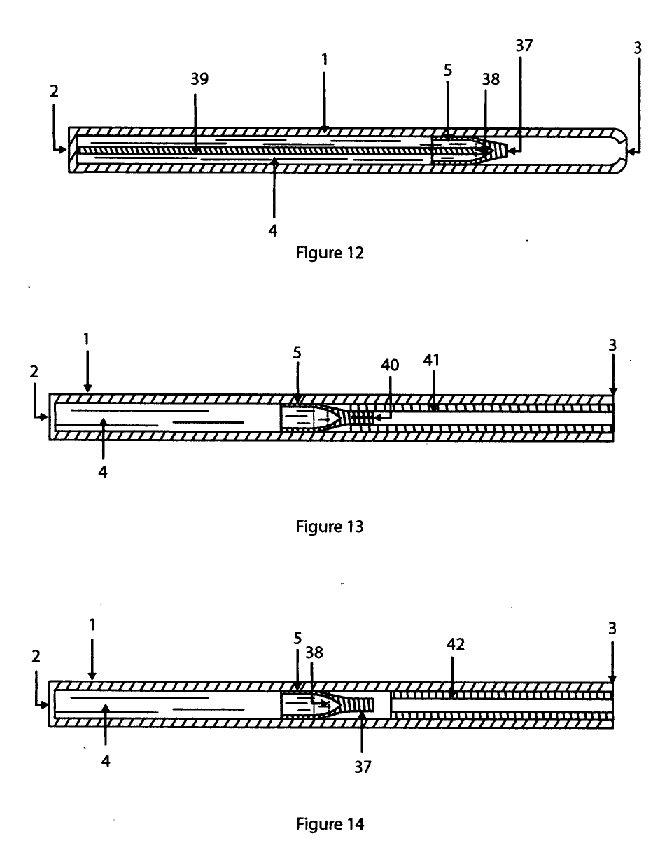

As shown in FIG. 12, the flexible elongated tubular housing 1 may be provided with a reduced opening at its open end 3 to capture the fractured sealed end 37 of the enclosed opening means 5. The reduced opening is provided with a profile that will maintain a fluid flow path even if the fractured sealed end 37 of the enclosed opening means 5 is lodged against it. This may be accomplished by forming the reduced opening in an elliptical shape or in any other shape that is different than the shape of the fractured sealed end 37 of the enclosed opening means 5.

When the flexible elongated tubular housing 1 has a small cross-section such that the substance 4 within it cannot be reliably released simply by opening the opening means 5 due to its surface tension, a guiding member 39 may be utilized to increase the capillary action and to overcome the surface tension of the substance 4 to release the substance 4 from the flexible elongated tubular housing 1 as shown in FIG. 12. The guiding member 39 may be affixed to the sealed end 2 of the flexible elongated tubular housing 1 and extends to near or slightly inside the open end of the opening means 5.

FIG. 13 shows another embodiment of the enclosed opening means. In this embodiment, the sealed end 40 of the enclosed opening means 5 has a circumference that provides one or more fluid flow path around the sealed end 40. The sealed end 40 may be formed in the shape of a geometric shape that is not a circle such as a triangle, a square, or a star shape such that when it is inserted into another tube 41 it will not seal off the tube 41 but would leave one or more fluid flow path between the sealed end 40 and the tube 41. The enclosed opening means 5 is used in a flexible elongated tubular housing 1 with a sealed end 2 and an open end 3. A substance 4 is enclosed within the flexible elongated tubular housing 1 near the sealed end 2. The enclosed opening means 5 is disposed inside the flexible elongated tubular housing 1 sealing the substance 4 within the flexible elongated tubular housing 1. Another tube 41 with two open ends is inserted into the open end 3 of the flexible elongated tubular housing 1 with one open end toward the sealed end 40 of the enclosed opening means 5 overlapping the sealed end 40 of the enclosed opening means 5.

FIG. 14 shows another embodiment of the enclosed opening means. In this embodiment, the enclosed opening means 5 is used in a flexible elongated tubular housing 1 with a sealed end 2 and an open end 3. A substance 4 is enclosed within the flexible elongated tubular housing 1 near the sealed end 2. An enclosed opening means 5 is disposed inside the flexible elongated tubular housing 1 sealing the substance 4 within the flexible elongated tubular housing 1. Another tube 42 with two open ends is inserted into the open end 3 of the flexible elongated tubular housing 1 with one open end disposed toward the sealed end 37 of the enclosed opening means 5.

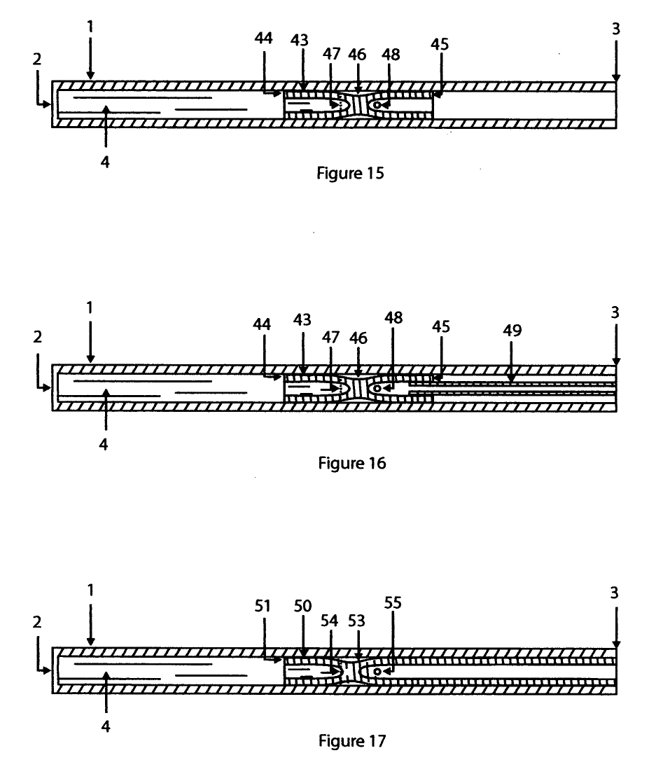

FIG. 15 shows another embodiment of the enclosed opening means comprising a tubular section 43 with two open ends 44, 45 and a seal 46 separating the two open ends 44, 45 disposed within the tubular section 43 between the two open ends 44, 45. The outside diameter of the tubular section 43 near the seal 46 is smaller than the outside diameter of the tubular section 43 near the two open ends 44, 45. A fracture line 47 is formed on one side of the seal 46 disposed at the section with the reduced outside diameter. An opening 48 such as a hole is formed on the other side of the seal 46 disposed at the section with the reduced outside diameter. The enclosed opening means 43 is inserted into a flexible elongated tubular housing 1 with a sealed end 2 and an open end 3 enclosing a substance 4 disposed near the sealed end 2. In the preferred embodiment, the enclosed opening means 43 is inserted such that the open end 44 with the fracture line 47 is disposed towards the substance 4 and the open end 45 with the opening 48 is disposed toward the open end 3 of the flexible elongated tubular housing 1. When the flexible elongated tubular housing 1 is bent at or near the fracture line 47 on the enclosed opening means 43 a fluid flow path is exposed leading from the substance 4 to the open end 3 of the flexible elongated tubular housing 1. The substance 4 will flow through the resulting opening from the fracture of the fracture line 47 into the area between the reduced outside diameter of the enclosed opening means 43 and the inside wall of the flexible elongated tubular housing 1 and then into the opening 48 on the other side of the seal 46 in the enclosed opening means 43 and out of the flexible elongated tubular housing 1.

FIG. 16 shows another embodiment of the enclosed opening means wherein a hollow tube 49 is inserted through the open end 3 of the flexible elongated tubular housing 1 and into the open end 45 of the enclosed opening means 43 shown in FIG. 15. In this embodiment, a more rigid hollow tube 49 may be used to increase the rigidity of the body of the flexible elongated tubular housing 1 near the open end 3.

FIG. 17 shows another embodiment of the enclosed opening means previously shown in FIG. 15 comprising a tubular section 50 with two open ends 51, 52 and a seal 53 separating the two open ends 51, 52 disposed within the tubular section 50 between the two open ends 51, 52. The outside diameter of the tubular section 50 near the seal 53 is smaller than the outside diameter of the tubular section 50 near the two open ends 51, 52. A fracture line 54 is formed on one side of the seal 53 disposed at the section with the reduced outside diameter. An opening 55 such as a hole is formed on the other side of the seal 53 disposed at the section with the reduced outside diameter. The enclosed opening means 50 is inserted into a flexible elongated tubular housing 1 with a sealed end 2 and an open end 3 enclosing a substance 4 disposed near the sealed end 2. In the preferred embodiment, the enclosed opening means 50 is inserted such that the open end 51 with the fracture line 54 is disposed towards the substance 4 and the open end 52 with the opening 55 is disposed toward the open end 3 of the flexible elongated tubular housing 1 wherein the open end 52 of the enclosed opening means 50 extends out of the flexible elongated tubular housing 1. When the flexible elongated tubular housing 1 is bent at or near the fracture line 54 on the enclosed opening means 50 a fluid flow path is exposed leading from the substance 4 to the open end 52 of the enclosed opening means 50. The substance 4 will flow through the resulting opening from the fracture of the fracture line 54 into the area between the reduced outside diameter of the enclosed opening means 50 and the inside wall of the flexible elongated tubular housing 1 and then into the opening 55 on the other side of the seal 53 in the enclosed opening means 50 and out of the open end 3 of the flexible elongated tubular housing 1.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An adjustable angle eye dropper comprising:
a flexible elongated tubular housing with a sealed end and an open end, a substance enclosed within the flexible elongated tubular housing near the sealed end, an enclosed opening means disposed inside the flexible elongated tubular housing sealing the substance within the flexible elongated tubular housing wherein the opening means is operated to release the substance from the flexible elongated tubular housing.

2. An adjustable angle eye dropper as in claim 1, wherein said enclosed opening means comprises of a cylinder with an outside diameter approximately that of the inside diameter of said flexible elongated tubular housing defining a fluid flow path from the substance to the open end of said flexible elongated tubular housing and an elongated protrusion that is separable from the cylinder extending from an end of the cylinder near the substance and sealing the fluid flow path.

3. An adjustable angle eye dropper as in claim 1, wherein said enclosed opening means comprises of a hollow cylindrical body with an open end disposed toward the substance and a sealed end with an elongated member extending therefrom to the open end of the flexible elongated tubular housing with a frangible section formed on the hollow cylindrical body near the sealed end of the hollow cylindrical body.

4. An adjustable angle eye dropper as in claim 1, wherein said enclosed opening means comprises of a plug that is fracturable at a fracture surface wherein said plug has one or more holes extending from each end of the plug that extend pass the fracture surface without passing through the other end of the plug wherein upon bending of the flexible elongated tubular housing at or near the plug the plug will fracture at said fracture surface and one or more fluid flow paths are formed leading from the substance to the open end of the flexible elongated tubular housing.

5. An adjustable angle eye dropper as in claim 1, wherein said enclosed opening means comprises of two circular discs with approximately the same cross-section as the inside diameter of the flexible elongated tubular housing and with sufficient thickness to prevent them from flipping inside the flexible elongated tubular housing wherein each circular disc has one or more holes through it positioned such that when the two circular discs are placed next to each other in the flexible elongated tubular housing the holes in each circular disc will be covered by the other disc thereby all the holes in both circular discs are sealed by the other circular disc and when the flexible elongated tubular housing is bent at or near the two circular discs the deformation of the flexible elongated tubular housing will cause the two circular discs to separate from each other and a fluid flow path will result.

6. An adjustable angle eye dropper as in claim 1, wherein said enclosed opening means comprises of an elongated cup with a diameter approximately that of the inside diameter of the flexible elongated tubular housing and a length that is longer than the diameter with a closed end terminate in an elongated protrusion with a fracture line formed around the elongated protrusion positioned along the elongated cup at a point where when the elongated protrusion is fractured an opening will be formed at the closed end of the elongated cup whereby said elongated cup is inserted into the flexible elongated tubular housing to seal the substance within the flexible elongated tubular housing wherein upon bending the flexible elongated tubular housing near the closed end of the elongated cup the elongated protrusion will fracture from the elongated cup at the fracture line and a fluid flow path is exposed to allow the substance to be released from the flexible elongated tubular housing.

7. An adjustable angle eye dropper comprising a flexible elongated tubular housing with a sealed end and an open end, an elongated member affixed to said sealed end, a substance enclosed within the flexible elongated tubular housing near the sealed end, an enclosed opening means disposed inside the flexible elongated tubular housing sealing the substance within the flexible elongated tubular housing wherein the opening means may be operated to release the substance from the flexible elongated tubular housing.

8. An adjustable angle eye dropper as in claim 7, wherein said enclosed opening means comprises of a cylinder with an outside diameter approximately that of the inside diameter of said flexible elongated tubular housing defining a fluid flow path from the substance to the open end of said flexible elongated tubular housing and an elongated protrusion that is separable from the cylinder extending from an end of the cylinder near the substance and sealing the fluid flow path.

9. An adjustable angle eye dropper as in claim 7, wherein said enclosed opening means comprises of a hollow cylindrical body with an open end disposed toward the substance and a sealed end with an elongated member extending therefrom to the open end of the flexible elongated tubular housing with a frangible section formed on the hollow cylindrical body near the sealed end of the hollow cylindrical body.

10. An adjustable angle eye dropper as in claim 7, wherein said enclosed opening means comprises of a plug that is fracturable at a fracture surface wherein said plug has one or more holes extending from each end of the plug that extend pass the fracture surface without passing through the other end of the plug wherein upon bending of the flexible elongated tubular housing at or near the plug the plug will fracture at said fracture surface and one or more fluid flow paths are formed leading from the substance to the open end of the flexible elongated tubular housing.

11. An adjustable angle eye dropper as in claim 7, wherein said enclosed opening means comprises of two circular discs with approximately the same cross-section as the inside diameter of the flexible elongated tubular housing and with sufficient thickness to prevent them from flipping inside the flexible elongated tubular housing wherein each circular disc has one or more holes through it positioned such that when the two circular discs are placed next to each other in the flexible elongated tubular housing the holes in each circular disc will be covered by the other disc thereby all the holes in both circular discs are sealed by the other circular disc and when the flexible elongated tubular housing is bent at or near the two circular discs the deformation of the flexible elongated tubular housing will cause the two circular discs to separate from each other and a fluid flow path will result.

12. An adjustable angle eye dropper as in claim 7, wherein said enclosed opening means comprises of an elongated cup with a diameter approximately that of the inside diameter of the flexible elongated tubular housing and a length that is longer than the diameter with a closed end terminate in an elongated protrusion with a fracture line formed around the elongated protrusion positioned along the elongated cup at a point where when the elongated protrusion is fractured an opening will be formed at the closed end of the elongated cup whereby said elongated cup is inserted into the flexible elongated tubular housing to seal the substance within the flexible elongated tubular housing wherein upon bending the flexible elongated tubular housing near the closed end of the elongated cup the elongated protrusion will fracture from the elongated cup at the fracture line and a fluid flow path is exposed to allow the substance to be released from the flexible elongated tubular housing.

13. An adjustable angle eye dropper comprising a flexible elongated tubular housing with a sealed end and an open end, an elongated member affixed to said sealed end, a substance enclosed within the flexible elongated tubular housing near the sealed end, an enclosed opening means with an elongated member disposed inside the flexible elongated tubular housing sealing the substance within the flexible elongated tubular housing with the elongated member extending towards the open end of the flexible elongated tubular housing wherein the opening means may be operated to release the substance from the flexible elongated tubular housing.

14. An adjustable angle eye dropper as in claim 13, wherein said enclosed opening means comprises of a cylinder with an outside diameter approximately that of the inside diameter of said flexible elongated tubular housing defining a fluid flow path from the substance to the open end of said flexible elongated tubular housing and an elongated protrusion that is separable from the cylinder extending from an end of the cylinder near the substance and sealing the fluid flow path.

15. An adjustable angle eye dropper as in claim 13, wherein said enclosed opening means comprises of a hollow cylindrical body with an open end disposed toward the substance and a sealed end with an elongated member extending therefrom to the open end of the flexible elongated tubular housing with a frangible section formed on the hollow cylindrical body near the sealed end of the hollow cylindrical body.

16. An adjustable angle eye dropper as in claim 13, wherein said enclosed opening means comprises of a plug that is fracturable at a fracture surface wherein said plug has one or more holes extending from each end of the plug that extend pass the fracture surface without passing through the other end of the plug wherein upon bending of the flexible elongated tubular housing at or near the plug the plug will fracture at said fracture surface and one or more fluid flow paths are formed leading from the substance to the open end of the flexible elongated tubular housing.

17. An adjustable angle eye dropper as in claim 13, wherein said enclosed opening means comprises of two circular discs with approximately the same cross-section as the inside diameter of the flexible elongated tubular housing and with sufficient thickness to prevent them from flipping inside the flexible elongated tubular housing wherein each circular disc has one or more holes through it positioned such that when the two circular discs are placed next to each other in the flexible elongated tubular housing the holes in each circular disc will be covered by the other disc thereby all the holes in both circular discs are sealed by the other circular disc and when the flexible elongated tubular housing is bent at or near the two circular discs the deformation of the flexible elongated tubular housing will cause the two circular discs to separate from each other and a fluid flow path will result.

18. An adjustable angle eye dropper as in claim 13, wherein said enclosed opening means comprises of an elongated cup with a diameter approximately that of the inside diameter of the flexible elongated tubular housing and a length that is longer than the diameter with a closed end terminate in an elongated protrusion with a fracture line formed around the elongated protrusion positioned along the elongated cup at a point where when the elongated protrusion is fractured an opening will be formed at the closed end of the elongated cup whereby said elongated cup is inserted into the flexible elongated tubular housing to seal the substance within the flexible elongated tubular housing wherein upon bending the flexible elongated tubular housing near the closed end of the elongated cup the elongated protrusion will fracture from the elongated cup at the fracture line and a fluid flow path is exposed to allow the substance to be released from the flexible elongated tubular housing.

19. An enclosed opening means for a flexible elongated tubular housing with a sealed end and an open end with a substance disposed near said sealed end comprising a cylinder with an open end and a sealed end with a sealing diameter provided around said cylinder that is approximately that of the inside diameter of the flexible elongated tubular housing and provides a seal to prevent the release of the substance from between the flexible elongated tubular housing and the opening means and with an outside diameter at the sealed end smaller than the inside diameter of the flexible elongated tubular housing and a fracture line provided near the sealed end of the cylinder at a location on the other side of the sealing diameter where the substance is disposed wherein said enclosed opening means is disposed in the flexible elongated tubular housing with the open end towards the substance thereby sealing the substance within the flexible elongated tubular housing.

20. An enclosed opening means as in claim 19, wherein said open end of said flexible elongated tubular housing has a smaller inside diameter than the flexible elongated tubular housing.

21. An enclosed opening means as in claim 19, wherein an elongated member is disposed near the sealed end of said flexible elongated tubular housing.

22. An enclosed opening means as in claim 19, wherein a tube with two open ends is inserted through the open end of said flexible elongated tubular housing.

23. An enclosed opening means as in claim 22, wherein an elongated member is disposed near the sealed end of said flexible elongated tubular housing.

24. An adjustable angle eye dropper for a flexible elongated tubular housing with a sealed end and an open end with a substance disposed near said sealed end comprising a tubular section with two open ends and a seal separating the two open ends disposed within the tubular section between the two open ends and an outside diameter of the tubular section near the seal that is smaller than an outside diameter of the tubular section near said two open ends with a fracture line provided on one side of the seal disposed at the section with the reduced outside diameter and an opening positioned on the other side of the seal disposed at the section with the reduced outside diameter wherein the enclosed opening means is affixed in the flexible elongated tubular housing such that the open end with the fracture line is disposed towards the substance and the open end with the opening is disposed toward the open end of the flexible elongated tubular housing.

25. An adjustable angle eye dropper as in claim 24, wherein an elongated member is disposed near the sealed end of said flexible elongated tubular housing.

26. An adjustable angle eye dropper as in claim 24, wherein a tube with two open ends is inserted through the open end of said flexible elongated tubular housing.

27. An adjustable angle eye dropper as in claim 26, wherein an elongated member is disposed near the sealed end of said flexible elongated tubular housing.
